(19) United States
(12) Patent Application Publication
(10) Pub. No.: US 2026/0215965 A1
(43) Pub. Date: Jul. 30, 2026

(54) RETINAL MEMBRANE DETECTION USING BRILLOUIN LIGHT SCATTERING

(71) Applicant: Alcon Inc., Fribourg (CH)

(72) Inventors: Gangjun Liu, Portland, OR (US); Lingfeng Yu, Rancho Santa Margarita, CA (US); Sumit Paliwal, Irvine, CA (US); Luyao Ma, Fort Worth, TX (US); Paul R. Hallen, Colleyville, TX (US)

(21) Appl. No.: 19/411,927

(22) Filed: Dec. 8, 2025

Related U.S. Application Data
(60) Provisional application No. 63/750,126, filed on Jan. 27, 2025.

(51) Int. Cl. A61F 9/007; A61B 3/00; A61B 3/13

(52) U.S. Cl. CPC A61F 9/007; A61B 3/0041; A61B 3/132

(57) ABSTRACT

An ophthalmic surgical system includes a biomechanical imaging device configured to perform measurements of an eye of a patient. The system further includes a controller configured to: receive Brillouin light scattering (BLS) data from the BLS imaging device; calculate material properties according to the BLS data; select a tissue type according to the material properties; and generating an output corresponding to the tissue type. The biomechanical imaging device may include a confocal Brillouin microscope or a BLS spectrometer and a light source coupled to a fiber optic probe. Tissue types may include a retinal membrane, type of retinal membrane, or membrane-free area of the retina.

### INTRODUCTION

The present disclosure relates generally to classifying tissue during ophthalmic surgery, such as retinal membranes.

The human eye receives light through a clear outer portion called the cornea and focuses the resulting image by way of an ocular crystalline lens onto the retina. The volume of the eye between the lens and the retina is occupied by a clear gel known as the vitreous. A thin film known as the internal limiting membrane (ILM) separates the retinal from the vitreous. Other pathological membranes may also form over the retina, such as an epiretinal membrane (ERM), diabetic membrane, drusen, or free-floating retina or retinal bleb in the case of a detached retina). Some conditions require removal of the ILM and an ERM may also need to be removed to restore visual acuity. Still other conditions require the vitreous to be removed. Many of these procedures may place stress on the retina. Accordingly, it is important to ensure that such procedures do not place undue stress on the retina and cause injury.

### SUMMARY

In certain embodiments, an ophthalmic surgical system includes a biomechanical imaging device configured to perform measurements of an eye of a patient. The system further includes a controller configured to: receive Brillouin light scattering (BLS) data from the biomechanical imaging device; calculate material properties according to the BLS data; select a tissue type according to the material properties; and generating an output corresponding to the tissue type.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example operating environment for providing ophthalmic treatments, in accordance with certain embodiments.

FIG. 2 illustrates an example membrane peeling operation for which tissue may be classified in accordance with certain embodiments.

FIG. 3 illustrates a system for classifying tissue of the retina in accordance with certain embodiments.

FIG. 4 illustrates an alternative system using a probe for classifying tissue of a retina of the retina in accordance with certain embodiments.

FIG. 5A illustrates a system for interfacing with a probe to measure simulated Brillouin scattering.

FIG. 5B illustrates an alternative system for interfacing with a probe to measure simulated Brillouin scattering.

FIG. 6 is a process flow diagram of a method for classifying tissue of the retina using simulated Brillouin scattering in accordance with certain embodiments.

FIGS. 7A, 7B, and 7C illustrate images for outputting classification of tissue of the retina in accordance with certain embodiments.

FIG. 8 illustrates an example computing device that implements, at least partly, one or more functionalities for classifying tissue of the retina, in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

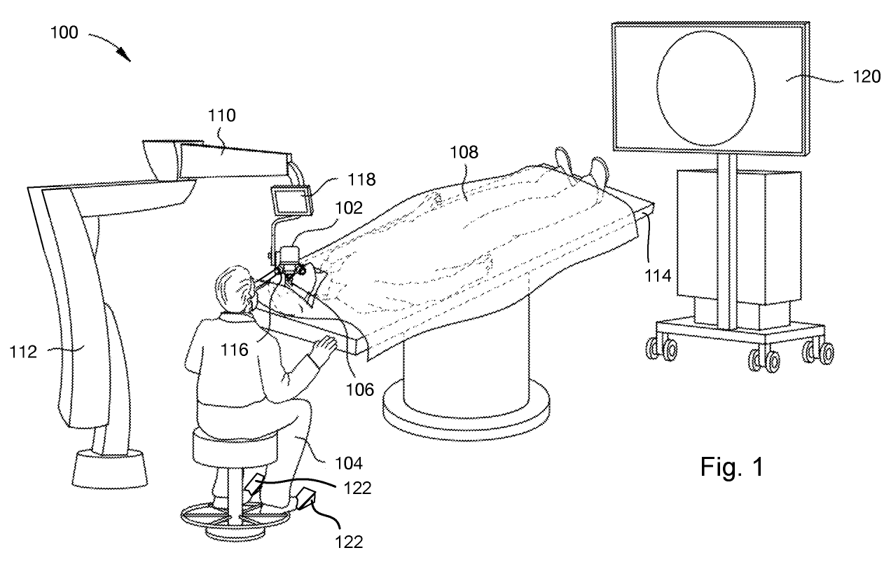

FIG. 1 illustrates an example operating environment including an ophthalmic surgical system 100 with which ophthalmic treatments may be performed. The ophthalmic surgical system 100 includes an ophthalmic microscope 102, used by a surgeon 104 to visualize structures on and in an eye 106 of a medical patient 108 in the field of view of the ophthalmic microscope 102. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing surgical microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and a broadband light source for capturing color (red, green, and blue) images and/or infrared images. The ophthalmic microscope 102 may, in certain embodiments, further include a multi-spectral imaging (MSI) device, and/or other type of imaging device. Digital images captured using the camera may be displayed on a display device within the ophthalmic microscope 102.

The ophthalmic microscope 102 may include two display devices that are viewable through binocular eyepieces 116 and that display images of the patient’s eye 106 captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may be additionally or alternatively displayed on one or more display devices. For example, the one or more display devices may include a display device 118 fastened to the overhead arm 110 above the ophthalmic microscope 102.

In order to relieve the surgeon 104 from the need to constantly look into the eye pieces 116 to obtain a stereoscopic view, the one or more display devices may also include a display device 120 that can be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

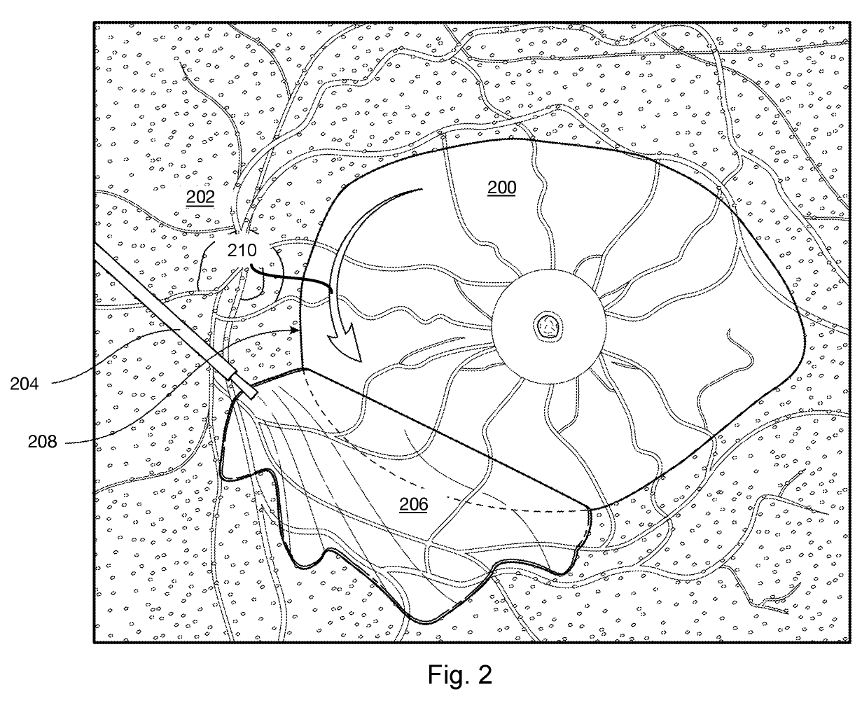

FIG. 2 the retina 200 may have various membranes 202 formed thereon that may be normal (e.g. the internal limiting membrane (ILM)) or pathological (epiretinal membrane (ERM), diabetic membrane, drusen, or retinal bleb). The surgeon 104 inserts an instrument 204 into the eye 106 and grasps the membrane 202. The instrument 204 may include forceps, a scraper, or other type of instrument. Prior to grasping, the instrument 204 or a different instrument may be used to pull on the membrane 202 and raise a flap that may then be grasped using the instrument 204. A portion 206 of the membrane within a region 208 (e.g., the macula of the retina 200) may then be peeled away from the retina 200. For example, the instrument 204 may be moved through a circular motion 210 to perform the peeling.

Many membranes are transparent and can be difficult to visualize. It can be difficult to distinguish between areas that have been peeled and those that remain to be peeled. The approach described herein provides an improved approach for identifying membranes on the retina.

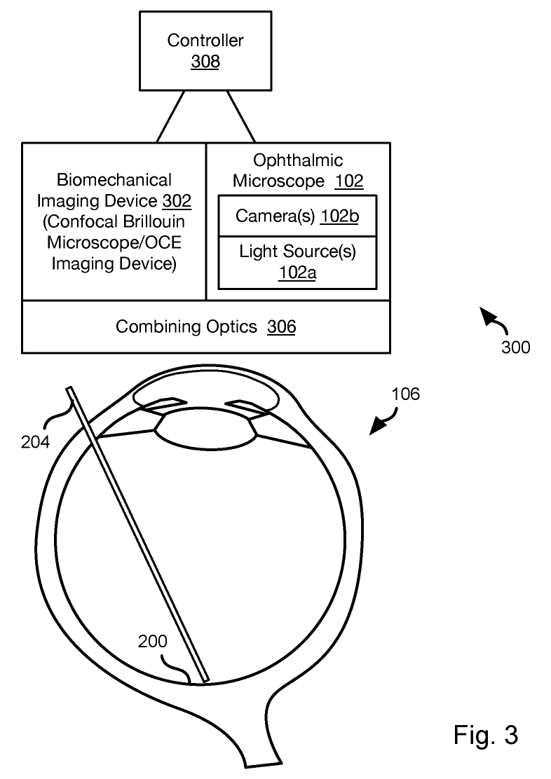

Referring to FIG. 3, in a first example system 300, biomechanical imaging device 302 is used along with the ophthalmic microscope 102. The biomechanical imaging device 302 may be implemented as, a Brillouin light scattering (BLS) imaging device, for example, a confocal Brillouin microscope. Alternatively, the biomechanical imaging device 302 maybe implemented as an optical coherence elastography (OCE) imaging device 302 can also be used along with the ophthalmic microscope 102.

The biomechanical imaging device 302 may use BLS to measure properties of a material by illuminating the material with light, which interacts with material and undergoes scattering. Material properties may include mechanical properties, such as elasticity and viscosity. As used herein, the wavelength band of light may be defined as the 3 dB bandwidth, e.g., a wavelength band such that all wavelength outside of the wavelength band are at least 3 dB lower than the peak amplitude of wavelength within the wavelength band. For example, the light may have a wavelength bandwidth of less than 100 picometers, less than 10 picometers or less than 1 picometers.

The biomechanical imaging device 302 and ophthalmic microscope 102 may be used with combining optics 306, e.g., one or more beam splitters that direct light from the biomechanical imaging device 302 into the eye 106 and direct at least a portion of newly generated scattered light back to the biomechanical imaging device 302. The combining optics 306 may direct at least a portion of reflected light into the ophthalmic microscope 102 such that the scattered or reflected light may be detected by a camera of the ophthalmic microscope 102. As an alternative, the biomechanical imaging device 302 and ophthalmic microscope 102 may be separate devices that have optical axes that are angled or offset relative to the optical axis of the eye 106.

A controller 308 may be coupled to the biomechanical imaging device 302 and ophthalmic microscope 102 in order to control operation thereof in a coordinated manner. For example, the ophthalmic microscope 102 may include one or more light sources 102a that may be turned off when the biomechanical imaging device 302 is in use to avoid interference with the detection of reflected light. The controller 308 may further augment images captured using one or more cameras 102b of the ophthalmic microscope 102 with tissue classifications determined using the biomechanical imaging device 302 as discussed in greater detail below.

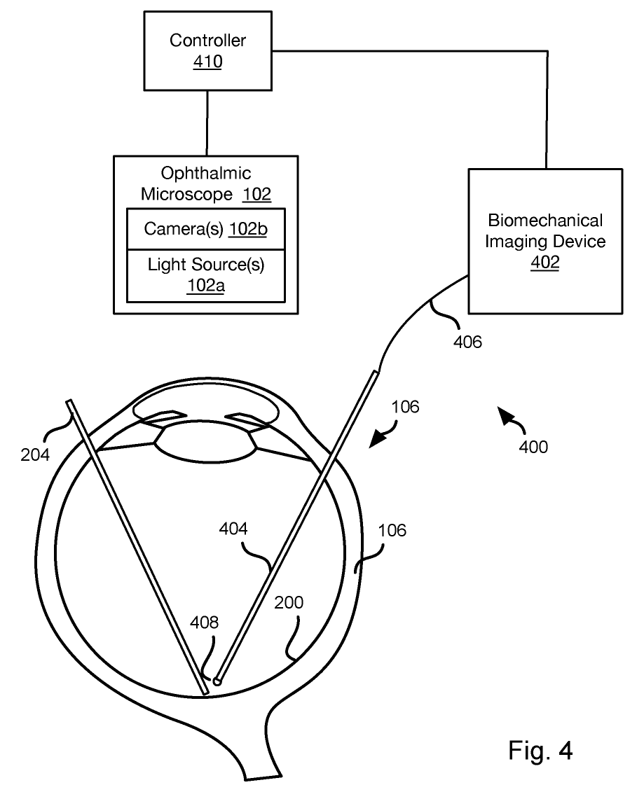

Referring to FIG. 4, in an alternative embodiment, a system 400 includes the ophthalmic microscope and may additionally include a biomechanical imaging device 402 connected by a fiber optic cable 406 to a probe 404. The probe 404 may conduct light between the fiber optic cable 406 and a distal end of the probe 404 that is inserted within the eye 106. The distal end may have a lens 408 formed or secured thereon that focuses light emitted from the probe 404 at a focal plane of the lens 408. The focal plane may be very close to the lens 408, such as within 100 micrometers, 50 micrometers, or 10 micrometers. The probe 404 may be positioned such that the focal plane of the lens 408 is on the retina 200 in order to measure material properties of a region of the retina 200. The region may be small, e.g., less than 100 micrometers, less than 50 micrometers, or less than 10 micrometers.

A controller 410 may be coupled to the biomechanical imaging device 402 and the ophthalmic microscope 102 and coordinate operation thereof. For example, the controller 410 may, for example, deactivate one or more light sources 102a of the ophthalmic microscope 102 when measurements are made using the biomechanical imaging device 402.

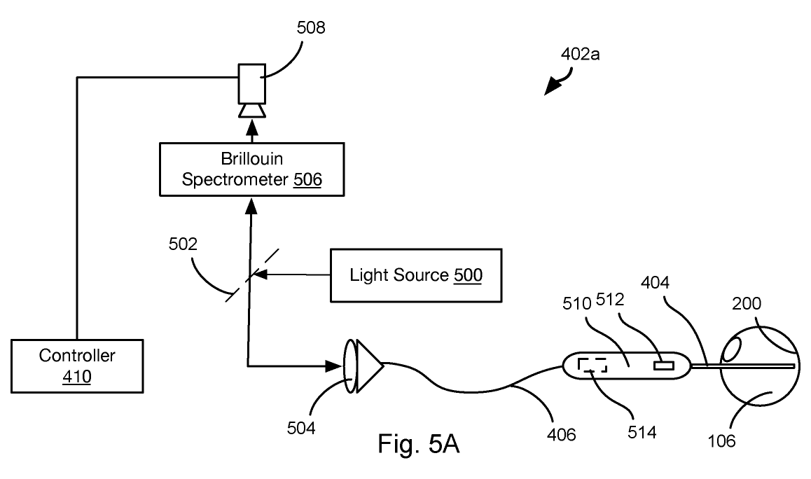

FIG. 5A illustrates an example biomechanical imaging device 402a that may be used to implement the biomechanical imaging device 402. The biomechanical imaging device 402 may include a light source 500 that directs light into the fiber optic cable 406. For example, a portion of the light from the light source 500 may pass through or be reflected by a beam splitter 502 and reach the fiber optic cable 406. The light may be conducted into the fiber optic cable 406 by a lens 504 or other interface. The light source 500 may be a laser and may emit continuous-wave or pulses, such as pulses having a duration of less than 100, 50, 10, or 5 picoseconds.

Light output from the fiber optic cable 406, e.g., scattered from within the eye 106, may be directed to the beam splitter 502 and a portion thereof may be reflected by or pass through the beam splitter 502 and reach a Brillouin spectrometer 506. The Brillouin spectrometer 506 may be a specialized spectrometer that is tuned to detect the miniscule frequency shifts caused by BLS. The Brillouin spectrometer 506 may include a detector 508, e.g., a camera or other photo detector capable of detecting the fringes created by the BLS. The Brillouin spectrometers 506 and detector 508 may be configured to detect wavelengths in a narrow band, e.g., less than 1 nanometer, less than 100 picometers, or less than 10 picometers. The Brillouin spectrometers 506 and detector 508 may be configured to detect wavelengths with a resolution of less than 1 picometer, less than 100 femtometers, less than 10 femtometers, or less than 1 femtometer.

The probe 404 may be integrated with the instrument 204. For example, the probe 404 may be configured to measure properties of material within a threshold distance (e.g., 100, 50, or 10 micrometers) of a distal end of the instrument 204. The probe 404 may be extensible independent of the instrument 204 (e.g., parallel to the instrument 204) to enable measurement independent of contact of the instrument 204 with the retina 200.

The probe 404 may be mounted to a handpiece 510 configured to be held in the hand of a surgeon. The handpiece 510 may include one or more user interface elements. For example, a button 512 mounted to the handpiece 510, a foot pedal 122, or other interface element may be coupled to the controller 410 and, when selected by the surgeon 104, causes the controller 410 to cause the biomechanical imaging device 402a to perform a measurement of material properties.

The handpiece 510 may include an output device 514. The output device 514 may include a light, speaker, haptic feedback device, or other type of output device 514. The output device 514 may be caused, by the controller 410, to produce an output based on material properties detected using the biomechanical imaging device 402a as described above.

In the embodiment of FIG. 5A, the fiber optic cable 406 may be a single core optical fiber (e.g., single mode fiber) such that measurements performed using the probe 404 are a single measurement of a region illuminated by the probe 404, e.g., at a focal point of the lens 408.

Referring to FIG. 5B, in some embodiments, the biomechanical imaging device 402a may be modified to implement the illustrated biomechanical imaging device 402b to perform measurements of material properties at a plurality of points within a region illuminated by the probe 404. In the embodiment of FIG. 5B, the fiber optic cable 406 may be a bundle of fibers and the lens 408 may focus an image at a focal plane thereof onto the bundle of fibers such that a two-dimensional image is transmitted through the fiber optic cable 406.

A scanner 520 may be interposed between the light source 500 and the fiber optic cable 406 and between the Brillouin spectrometer 506 and the fiber optic cable 406. For example, the scanner 520 may be interposed between the beam splitter 502 and the fiber optic cable 406. For example, a scanned beam output by the scanner 520 may be scanned onto the lens 504.

The scanner 520 may be a scanning mirror, such as a mirror capable of scanning in one angular dimension or two angular dimensions. For example, the scanner 520 may include one, two, or more Galvo mirrors, a micro electromechanical system (MEMS) mirror or pair of MEMS mirrors, or other type of scanning mirror or set of scanning mirrors. The scanner 520 may scan in a circular or spiral pattern conforming to the circular shape of the fiber optic cable 406, a back-and-forth pattern, or other scanning pattern such as a raster pattern. Where the scanner 520 is one-dimensional, the scanning pattern may be a line. The scanner 520 may scan a beam output by the light source 500 across the lens 504 or a cut plane of the fiber optic cable 406 such that the beam is selectively input into individual fibers of the bundle of fibers forming the fiber optic cable 406. Likewise, light scattered from within the eye 106 may be collected by the fiber optic cable 406 and descanned onto an optical axis of the Brillouin spectrometer 506. Outputs of the detector 508 may be related to the position of the scanner 520 in order to assemble outputs of the detector 508 into an image. In some embodiments, a filter 500a may be positioned between the scanner 520 and the Brillouin spectrometer 506, such as between the beam splitter 502 and the Brillouin spectrometer 506. The filter 500a may be a narrowband filter, e.g., having a 3 dB passband of less than 1 nanometer, less than 100 picometers, less than 10 picometers, or less than 1 picometer.

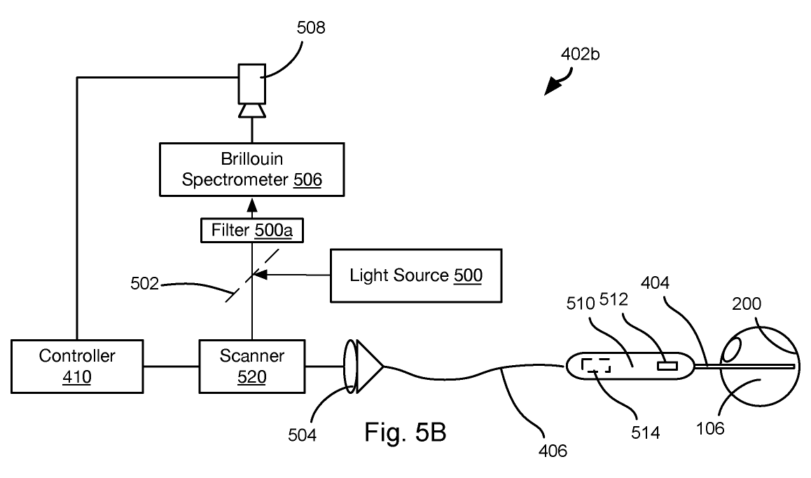

FIG. 6 illustrates a method 600 that may be performed by a controller 308, 410 according to the embodiments disclosed herein. The method 600 may include capturing, at step 602, a fundus/wideband image. A fundus image may be captured with the retina 200 illuminated with wideband, e.g., white, light. The fundus image may be captured with one or more cameras 102b of the ophthalmic microscope 102. The fundus image may be pair of images captured with a pair of cameras 102b, a three-dimensional image derived from a pair of images, or a volumetric image derived from the pair of images.

The method 600 may include capturing, at step 604, BLS data. Capturing BLS data may include capturing an image of the retina using the biomechanical imaging device 302, collecting a BLS measurement for a region of the retina using a biomechanical imaging device 402a, or capturing an image of the retina using the biomechanical imaging device 402b.

The method 600 may include calculating, at step 606, material properties for the BLS data from step 604. In particular, for each data point (e.g., pixel in an image or a single measurement for the biomechanical imaging device 402a), a material property for that data point may be calculated. For example, the material property may be a Young’s modulus that is calculated based on a detected frequency shift of the scattered light from the spectrometer.

The method 600 may include classifying, at step 608, the material property corresponding to each data point. In particular, for the material property calculated for a data point, a tissue having a range of values corresponding to that material property may be selected from a plurality of possible tissues at step 608. For example, a membrane may be softer (lower Young’s modulus of elasticity) than the retina. Other tissue layers, such as nerve fiber layer or Ganglion cell layer may also be classified in a like manner. Accordingly, moduli of elasticity in a first range may be deemed to correspond to a membrane whereas moduli of elasticity in a second range higher than the first range may be deemed to correspond to the other retina layer. The classification of step 608 may be more specific, e.g., different types of membranes (ILM, ELM, diabetic membrane, retinal bleb) may have different material properties such that a material property calculated for a data point may be mapped to a membrane having a range of material properties including the material property.

The method 600 may include generating an output that communicates the classification of step 608 to the surgeon 104. The output may include generating an output using a haptic feedback device, flashing light, speaker, or other output device. For example, if the output of the classification of step 608 is a membrane to be peeled, an output may be generated to communicate this fact. If the output of the classification of step 608 is the retina without a membrane, then no output is generated. In other embodiments, the output is a warning such that an output is generated if the classification indicates the retina without a membrane and an output is not otherwise generated.

In the illustrated embodiment, the method 600 may include generating, at step 610, an augmented image and displaying, at step 612, the augmented image. The augmented image may include at least a portion of the wideband image from step 602 having additional information superimposed thereon. The augmented image may be displayed on one of the display devices 118, 120, in a display internal to the ophthalmic microscope, or on some other display device.

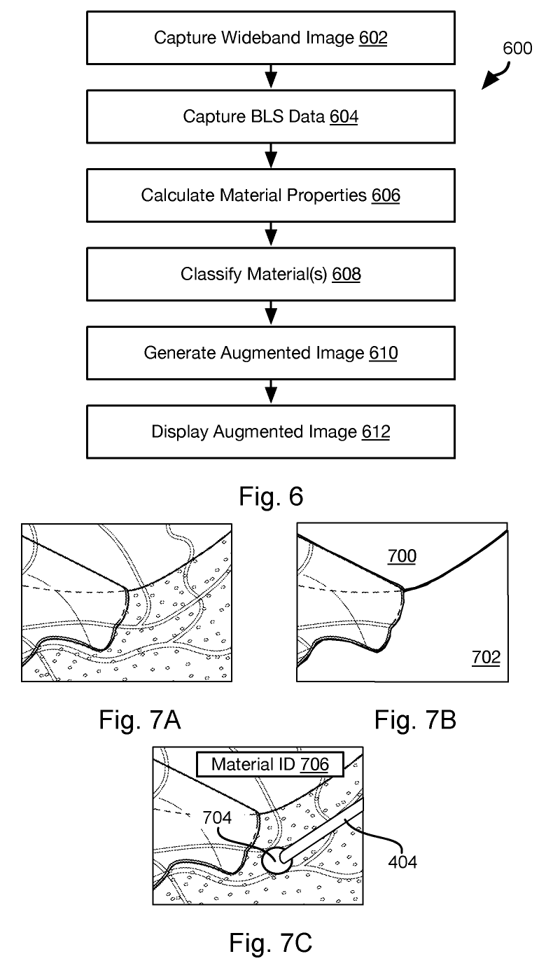

For example, FIG. 7A illustrates an example wideband image as captured using the ophthalmic microscope 102. FIG. 7B includes the wideband image having one or more regions 700 and 702 that are modified relative to the wideband image visually distinguished from one or both of (a) other portions of the wideband image and (b) one another. For example, region 700 corresponds to a portion of the retina 200 that is covered by a membrane and region 702 corresponds to a region of the retina 200 that has been peeled.

FIG. 7C illustrates another example augmented image. A region for which BLS data was collected may include a label 704. The label 704 may be positioned on the wideband image at a location corresponding to where the BLS data was collected. For example, a representation of the probe 404 may be identified in the wideband image and the location for the label 704 may be presumed to be at the representation of the distal end of the probe 404. The label 704 may include a color, texture, shape, symbol, alphanumeric character, or other visual attribute that corresponds to the classification of step 608. For example, red may indicate that the location of the label 704 has already been peeled and green may indicate that peeling should be performed at the location of the label 704. The label 704 may include an image captured using the biomechanical imaging device 402b. The image may be a false color image communicating the classification from step 608, such as in the same manner in which the regions 700, 702 are visually distinguished from one another in FIG. 7B.

The augmented image may additionally or alternatively include a label 706 that is not placed based on the estimated location at which a measurement was made. The label 706 may include text, one or more symbols, a color, or other visual indicator indicating an identifier of the tissue selected at step 608.

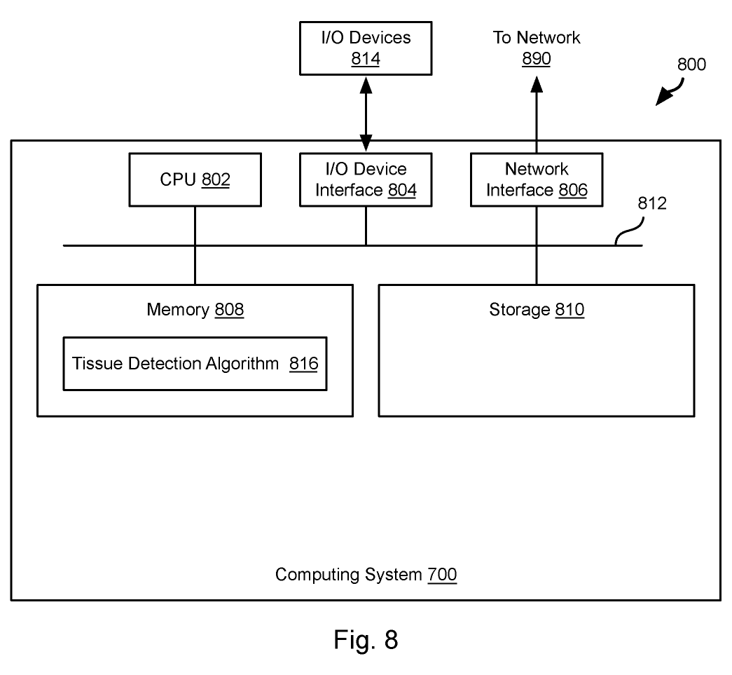

FIG. 8 illustrates an example computing system 800. The ophthalmic microscope 102, display devices 118, 120, and/or controllers 308, 410 may incorporate a computing device having some or all of the attributes of the computing system 800.

As shown, computing system 800 includes a central processing unit (CPU) 802, one or more I/O device interfaces 804, which may allow for the connection of various I/O devices 814 (e.g., keyboards, displays, mouse devices, pen input, etc.) to computing system 800, network interface 806 through which computing system 800 is connected to network 890, a memory 808, storage 810, and an interconnect 812.

CPU 802 may retrieve and execute programming instructions stored in the memory 808. Similarly, CPU 802 may retrieve and store application data residing in the memory 808. The interconnect 812 transmits programming instructions and application data, among CPU 802, I/O device interface 804, network interface 806, memory 808, and storage 810. CPU 802 is included to be representative of a single CPU, multiple CPUs, a single CPU having multiple processing cores, and the like.

Memory 808 is representative of a volatile memory, such as a random access memory, and/or a nonvolatile memory, such as nonvolatile random access memory, phase change random access memory, or the like. As shown, memory 808 may store executable code implementing a tissue detection algorithm 816, such as an algorithm implementing the method 600.

Storage 810 may be non-volatile memory, such as a disk drive, solid state drive, or a collection of storage devices distributed across multiple storage systems.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An ophthalmic surgical system comprising:
a biomechanical imaging device configured to perform measurements of an eye of a patient; and
a controller configured to:
receive Brillouin light scattering (BLS) data from the BLS imaging device;
calculate material properties according to the BLS data;
select a tissue type according to the material properties; and
generating an output corresponding to the tissue type.

2. The ophthalmic surgical system of claim 1, wherein the biomechanical imaging device is configured to image a retina of the eye of the patient.

3. The ophthalmic surgical system of claim 1, wherein the biomechanical imaging device is a Brillouin confocal microscope.

4. The ophthalmic surgical system of claim 1, wherein the biomechanical imaging device includes a light source, a Brillouin spectrometer, and a detector, the biomechanical imaging device configured to direct light from the light source onto the eye of the patient and direct a scattered portion of the light onto the Brillouin spectrometer, the detector configured to detect fringes in an output of the Brillouin spectrometer.

5. The ophthalmic surgical system of claim 4, further comprising a fiber optic cable configured to conduct the light from the light source to the eye of the patient and direct the scattered portion to the biomechanical imaging device.

6. The ophthalmic surgical system of claim 5, wherein the fiber optic cable includes a bundle of optical fibers and the biomechanical imaging device includes a scanner interposed between the fiber optic cable and the light source.

7. The ophthalmic surgical system of claim 5, wherein the fiber optic cable is secured to a probe configured to insert within the eye of the patient.

8. The ophthalmic surgical system of claim 1, wherein the controller is configured to generate the output using at least one of a light, speaker, or haptic feedback device.

9. The ophthalmic surgical system of claim 1, further comprising an ophthalmic microscope and a display device, the controller further configured to:
receive an image from the ophthalmic microscope;
label regions in the image according to the tissue type to obtain an augmented image; and
output the augmented image to the display device.

10. The ophthalmic surgical system of claim 1, wherein the tissue type is one of a retinal membrane and retina uncovered by a retinal membrane.

11. A method comprising:
receiving, by a controller, Brillouin light scattering (BLS) data from a biomechanical imaging device measuring an eye of a patient;
calculating, by the controller, material properties according to the BLS data;
selecting, by the controller, a tissue type according to the material properties; and
generating, by the controller, an output corresponding to the tissue type.

12. The method of claim 11, wherein the BLS data includes one or more measurements of a retina of the eye of the patient.

13. The method of claim 11, wherein the biomechanical imaging device is a BLS confocal microscope.

14. The method of claim 11, wherein the biomechanical imaging device includes a light source, a Brillouin spectrometer, and a detector, the biomechanical imaging device configured to direct light from the light source onto the eye of the patient and direct a scattered portion of the light from the light source onto the Brillouin spectrometer, the detector configured to detect fringes in an output of the Brillouin spectrometer.

15. The method of claim 14, wherein the biomechanical imaging device includes a fiber optic cable configured to conduct the light from the light source to the eye of the patient and direct the scattered portion to the biomechanical imaging device.

16. The method of claim 15, wherein the fiber optic cable includes a bundle of optical fibers and the biomechanical imaging device includes a scanner interposed between the fiber optic cable and the light source.

17. The method of claim 15, wherein the fiber optic cable is secured to a probe configured to insert within the eye of the patient.

18. The method of claim 11, wherein generating the output corresponding to the tissue type comprises generating the output using at least one of a light, speaker, or haptic feedback device.

19. The method of claim 11, further comprising:
receiving, by the controller, an image from an ophthalmic microscope;
labelling, by the controller, regions in the image according to the tissue type to obtain an augmented image; and
outputting, by the controller, the augmented image to a display device.

20. The method of claim 11, wherein the tissue type is one of a retinal membrane and retina uncovered by a retinal membrane.
